(19) United States
(12) Patent Application Publication
(10) Pub. No.: US 2023/0201504 A1
(43) Pub. Date: Jun. 29, 2023

(54) SYSTEM AND METHOD FOR GENERATING PATIENT-SPECIFIC VENTILATION SETTINGS BASED ON LUNG MODELING

(71) Applicant: Vyaire Medical, Inc., Mettawa, IL (US)

(72) Inventor: Christopher M. Varga, Palm Coast, FL (US)

(21) Appl. No.: 17/922,773

(22) PCT Filed: May 1, 2020
(86) PCT No.: PCT/US2021/030508

§ 371 Date: Nov. 1, 2022

Related U.S. Application Data
(60) Provisional application No. 63/019,218, filed on May 1, 2020.

(51) Int. Cl. A61M 16/00; G16H 40/40

(52) U.S. Cl. CPC A61M 16/026; G16H 40/40; A61M 2230/46

(57) ABSTRACT

The disclosed system and method generates a lung model based on patient data, and determines patient-specific ventilation settings for adjusting an operation of a ventilator. In this manner, the subject technology simulates flow in the lungs of COVID-19 patients to provide insights that guides improved ventilation and/or respiratory treatment strategies

This is a National Stage Application filed under 35 U.S.C. 371 based on International Patent Application No. PCT/US2021/030508, filed on May 3, 2021, which claims priority to U.S. Patent Application No. 63/019,218, filed on May 1, 2020, the disclosures of which are incorporated herein by reference in their entireties.

### BACKGROUND FIELD

The subject technology addresses deficiencies commonly encountered in hospital care with regard to assessing conditions of ventilated patients and adjusting ventilation parameters to stabilize such patients.

### SUMMARY

Evolving understanding of the pathophysiology of respiratory deterioration in COVID-19 patients indicates a unique behavior of the lungs which is not consistent with observations in previously encountered viral diseases and common lung conditions. Coupled with observed high mortality rates in intubated mechanically-ventilated COVID-19 patients, this suggests the need for a practical understanding of the associated pulmonary mechanics to guide respiratory therapy. While this is not easily accomplished in vivo due to the disease progression, it can be efficiently analyzed in silico.

The subject technology simulates flow in the lungs of COVID-19 patients to provide insights that may guide improved ventilation and/or respiratory treatment strategies. A method is disclosed wherein a computed tomography (CT) scan of the patient's lungs is converted into a computational model to simulate ventilation using computational fluid dynamics (CFD) and fluid-structure interaction (FSI). The subject technology further determines flow-related phenomena that contribute to ventilation strategies which improve COVID-19 lungs. For comparison purposes, simulations are performed in lung models obtained from healthy subjects as well as patients with non-COVID-19 pneumonia and/or ARDS. These flow simulations are utilized to evaluate and determine the best mode of ventilation for individual COVID-19 patients along with the most appropriate associated settings (e.g. FiO2, tidal volume, peak inspiratory pressure, positive end expiratory pressure (PEEP), respiratory rate, rise time) to maximize oxygenation while minimizing lung damage, and to optimize ventilator weaning. With a sufficient number of patient scans and associated clinical data, machine-learning algorithms are trained to make the above-mentioned predictions quickly. The implementation of such a workflow provides data-driven recommendations for patient-specific ventilation settings, enabling clinicians to optimize individual patient outcomes in the battle against coronavirus and future pandemics.

According to various implementations, the disclosed system includes one or more processors and a memory. The memory includes instructions stored thereon that, when executed by the one or more processors, cause the one or more processors to perform operations for performing a method of generating patient-specific ventilation parameters based on a lung model simulation, for adjustment of operating parameters of a ventilator. The method includes receiving diagnostic information for a patient, the diagnostic information including a first image scan and a second image scan of the patient's lungs; determining a lung model of a lung airway tree and lung margins for the patient; assigning uniform initial compliance to spherical volumes; executing a three-dimensional simulation on the first scan at a baseline ventilation condition, allowing the lung module to expand until a total volume of the lung model matches a measured air volume detected from the second scan; recording a volume change of each sphere; assigning each terminal branch in the segmented airway model to a fixed location on the lung margin of the first scan; connecting each associated point on the lung margin of the first scan with a location on the lung margin of the second scan using a surface normal value; recording a distance between associated points that are associated with each terminal branch; converting the volume change of each sphere into a linear displacement of its apex and compare to the associated displacement of the lung margin from the first scan to the second scan; using a predetermined one-dimensional model, adjust the compliance of each spherical volume until all displacements match correctly with measured displacements of the lung margin associated with each terminal branch; running a three-dimensional simulation with regional compliances to validate results of a one-dimensional model; correlating the diagnostic information or determined models with candidate results to determine optimal ventilation parameters; and adjusting, based on the determined optimal ventilation parameters, one or more current operating parameters of the ventilator. Other aspects include corresponding systems, apparatuses, and computer program products for implementation of the computer-implemented method.

Further aspects of the subject technology, features, and advantages, as well as the structure and operation of various aspects of the subject technology are described in detail below with reference to accompanying drawings.

### DESCRIPTION OF THE FIGURES

Various objects, features, and advantages of the present disclosure can be more fully appreciated with reference to the following detailed description when considered in connection with the following drawings, in which like reference numerals identify like elements. The following drawings are for the purpose of illustration only and are not intended to be limiting of this disclosure, the scope of which is set forth in the claims that follow.

FIG. 1 depicts an example digital lung model and flow simulation results (left: pressure distribution, right: air flow velocity magnitude), according to various aspects of the subject technology.

FIG. 2 depicts an example of lung geometries obtained from HRCT scans, according to various aspects of the subject technology.

FIG. 3 depicts an example schematic modeling approach for alveolar beds, according to various aspects of the subject technology.

FIG. 4 is a block diagram illustrating an example system for generating patient-specific ventilation settings based on lung modeling, and for adjusting an operation of a ventilator, including a ventilation device, one or more management devices, according to certain aspects of the subject technology.

FIGS. 5A and 5B depict an example flow chart of a method of generating patient-specific ventilation settings based on lung modeling, and for adjusting an operation of a ventilator, according to aspects of the subject technology.

FIG. 6 is a conceptual diagram illustrating an example electronic system for generating patient-specific ventilation settings based on lung modeling, and for adjusting an operation of a ventilator, according to aspects of the subject technology.

### DESCRIPTION

While aspects of the subject technology are described herein with reference to illustrative examples for particular applications, it should be understood that the subject technology is not limited to those particular applications. Those skilled in the art with access to the teachings provided herein will recognize additional modifications, applications, and aspects within the scope thereof and additional fields in which the subject technology would be of significant utility.

The subject technology comprises a computer-enabled system which integrates and weighs inputs obtained from lung models and mechanical ventilator data, alongside additional inputs obtained from integrated measurement devices and components. Objective patient physiological attributes and related measurements are obtained to produce lung models. In some implementations, the assessment system of the subject technology may use these inputs to provide patient-specific optimal ventilation settings, modes, or options.

The subject technology includes simulating flow in the lungs of COVID-19 patients to suggest appropriate patient-specific ventilation treatment strategies for clinicians managing coronavirus, and for adjusting an operation of a ventilator. In this regard, the subject technology includes:
obtaining scans and creating patient-specific lung models for both COVID-19 and non-COVID-19 patients;
simulating different ventilatory flows in said lung models;
evaluating the impact of said different ventilatory flows on the structure/function of the lungs;
correlating results of these simulations to clinical and patient-specific physiological parameters and outcomes;
recommending patient-specific optimal ventilation settings based on simulation data.

The objective of this disclosure is to provide improved treatment strategies for COVID-19 patients as well as improved understanding of ventilator interactions with common lung disease. Specifically the inventors seek to create guidance for patient-specific ventilation modes and settings to optimize oxygenation, mitigate lung injury and expedite weaning.

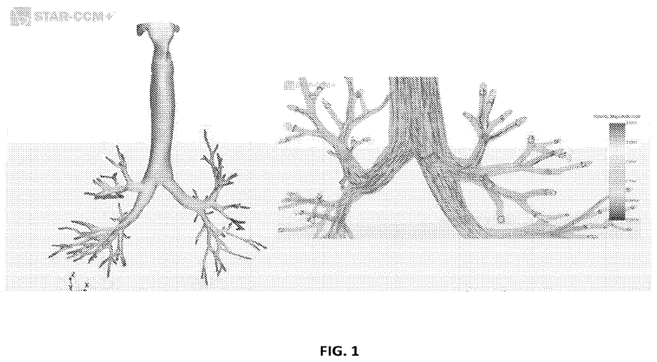

FIG. 1 depicts an example digital lung model and flow simulation results (left: pressure distribution, right: air flow velocity magnitude), according to various aspects of the subject technology. According to various implementations, the subject technology includes converting high-resolution computed tomography (HRCT) scans of patient lungs into computational models suitable for simulation of ventilation using computational fluid dynamics (CFD) and fluid structure interaction (FSI). Detailed CFD and FSI simulations are subsequently performed to characterize each lung model at clinically-relevant flow conditions. FIG. 1 contains a preliminary example of the flow mapping in a digital patient lung model generated from an HRCT scan.

### Problem/Solution

Mechanical ventilation of COVID-19 patients has proven difficult for clinicians to date during the coronavirus pandemic. Unexpected outcomes and high mortality rates have been encountered when following established and historically effective protocols developed for management of pneumonia and acute respiratory distress syndrome (ARDS). As clinicians continue to learn more about COVID-19, identification of different phenotypes and alternative ventilation strategies are emerging, however, it nevertheless remains difficult to determine a priori which ventilation or respiratory support approach is best for the individual patient. Thus, there is a need for patient-specific ventilation guidance. The disclosed approach has the potential to provide improved outcomes through simulation-based insights and patient-specific ventilation strategies. Improved outcomes include improved oxygenation, reduced lung injury, and shorter duration of ventilation. The manifestation of these improved patient outcomes also translate to a reduction in the overall burden of COVID-19 patients on hospital resources as well as ICU-bed availability and ventilator utilization. Furthermore, the resultant tools and strategies should beneficially be adaptable to future respiratory pandemics as well as common lung disease.

### Background

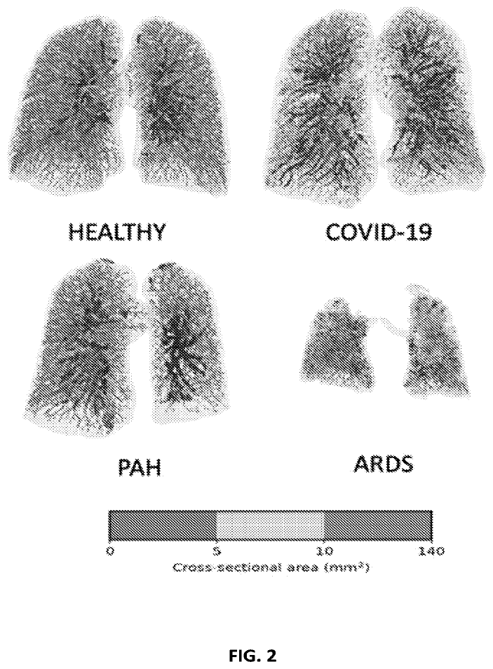

Evolving understanding of the pathophysiology of respiratory deterioration in COVID-19 patients indicates a unique behavior of the lungs which is not fully consistent with observations in traditional pneumonia and acute respiratory distress. Identification of multiple phenotypes has made it challenging to manage patients using historical ventilation protocols. High-mortality rates and extended ventilator length-of-stay have been unfortunate hallmarks of COVID-19 to date. Recommendations for ventilation modalities (e.g. non-invasive vs. invasive) based on limited and inconsistent data have additionally made it difficult for clinicians to determine the most appropriate course of action for these patients. Selection of a particular mechanical ventilation mode and associated settings traditionally follows from clinician experience, rules of thumb, and established protocols. Clinicians generally have limited to no visibility of the dynamic performance of the lungs in terms of localized flows and pressures, ventilation and perfusion matching, and the potential contribution of flow and pressure to lung damage in specific regions of the respiratory tree. Measuring bulk flow and gas composition entering and exiting a patient's respiratory tract does not provide any data or insight about flows and gas compositions in particular bronchi or bronchioles or regions of the alveolar bed which may be critical to understanding the nuances of the individual patient's respiratory performance or status. There is a clear need for a tool or method to provide patient-specific or individualized lung flow data for COVID-19 patients to guide appropriate mechanical ventilation strategies (and settings) or alternative respiratory support. Computational fluid dynamic (CFD) simulations of flow within digital models of human lungs can provide the necessary data to evaluate, understand and visualize the effects of various ventilation strategies on the respiratory response of COVID-19 patients. Subject-specific geometries and boundary conditions are established from high-resolution CT scans and enable the creation of near-exact digital replicas (or digital twins) of an individual patient's lungs. FIG. 2 shows an example of the use of high-resolution CT scans of COVID-19, healthy, ARDS, and pulmonary arterial hypertension (PAH) lungs to compare vasoconstriction in small blood vessels.

### Objectives and Phases

The subject technology simulates flow in the lungs of COVID-19 patients to provide insights that may guide improved ventilation and/or respiratory treatment strategies. The subject technology includes a system that determines flow-related phenomena that contribute to ventilation strategies which improve COVID-19 lungs. For comparison purposes, additional simulations are performed in lung models obtained from healthy subjects as well as patients with non-COVID-19 pneumonia and/or ARDS. These flow simulations are utilized to evaluate and determine the best mode of ventilation for individual COVID-19 patients along with the most appropriate associated settings (e.g. FiO2, tidal volume, peak inspiratory pressure, positive end expiratory pressure (PEEP), respiratory rate, rise time). Implementation of workflows based on such simulations and insights will enable ventilator manufacturers to suggest optimal ventilation settings for individual patients to improve therapy and outcomes in the current COVID-19 crisis as well as future pandemics. These workflows may be integrated into analytics platforms and related software. The proposed phases and associated steps for the proposed project are as follows:

### Phase One

Goal: Establish Individualized (Patient-Specific) Guidelines for Ventilation of COVID-19 Lungs

### Steps

1. Obtain HRCT Scans for Ten COVID-19 and Ten Non-COVID-19 Lungs (Healthy, ARDS) with Associated Ventilator and Patient Data.
2. Manually Segment and Create Digital Models of Lungs for CFD/FSI Simulations (e.g. from the HRCT scans).
3. Perform CFD/FSI Breathing Simulations of Various Ventilation Modes and Settings (e.g. using the digital models).
4. Evaluate Results (e.g. of the CFD/FSI Breathing Simulations), Validate and Correlate Simulations with Ventilator and Patient Physiologic Data.
5. Establish Recommended Settings for Ventilation of COVID-19 Lungs with Clinicians Based on Simulations and/or evaluation of results.

### Phase Two

Goal: Incorporate Guidelines/Workflow for Ventilation of COVID-19 Lungs into Medical Ventilator Analytics Software

### Steps

1. Create Fully-Automated Segmentation Algorithm to Create Digital Models of Lungs from HRCT scans for CFD/FSI Simulations
2. Develop and Train Machine-Learning Algorithm Using HRCT Scans (100s) and CFD/FSI Simulations to Predict Regional Lung Properties Based on Matched Models with Comparable Lung Morphology and Associated Patient Data
3. Create Software Module with Simplified Workflow for Accepting HRCT Scan Files and Outputting Recommended Settings for Ventilation of COVID-19 Lungs
4. Incorporate Software Module into a Medical Ventilation Platform (e.g., a Respiratory Knowledge Portal)

### Technical Approach

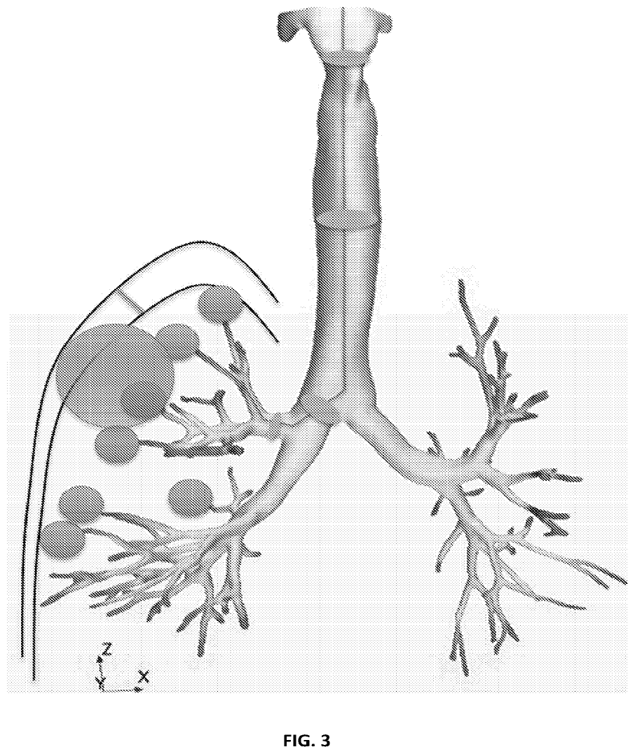

FIG. 3 depicts an example schematic modeling approach for alveolar beds, according to various aspects of the subject technology.

As will be described further, the disclosed technical approach for modeling patient lung performance and determining patient-specific regional lung compliances involves the following steps.
A. Obtain two CT scans of a patient's lungs (Scan #1 is taken at a breath-hold at the end of exhalation and Scan #2 is taken at a breath-hold at the end of inhalation).
B. Create three-dimensional (3D) models of the lung airway tree and lung margins (envelope perimeter) based on the obtained CT scans.
C. Attach an equal spherical compliant volume to the end of each (e.g. modeled) terminal lung branch (bronchiole) as shown in FIG. 3 to represent the alveolar bed fed by that branch. The initial volume of each sphere is determined by the measured air volume from Scan #1 minus the volume of the segmented airway tree, divided by the total number of terminal branches.
D. Assign a uniform initial compliance to all spherical volumes.
E. Run 3D FSI simulations on (the model of) Scan #1 at baseline ventilation conditions, allowing the lung model to expand until the total volume matches the measured air volume from Scan #2.
F. Record the volume change of each sphere (ratio Vol2/Vol1 per sphere)
G. Assign each terminal branch in the segmented airway model to a fixed location on the lung margin of Scan #1 (location determined by extending branch to nearest location on perimeter).
H. Connect each associated point on the lung margin of Scan #1 with a location on the lung margin of Scan #2 via surface normal (shown as green Line in FIG. 3).
I. Record the distance between associated points (i.e. length of Green line) associated with each terminal branch (terminal bronchiole).
J. Convert the volume change of each sphere into the linear displacement of its apex and compare to the associated displacement of the lung margin from Scan #1 to Scan #2.
K. Optimization: Using a 1D-model, adjust the compliance of each spherical volume until all displacements match correctly with the measured displacements of the lung margin associated with each terminal branch—we now have the regional compliance map for the lungs.
L. Run 3D FSI simulation with regional compliances to validate results of 1D-model.
M. Run simulations with 1D-model to establish appropriate ventilation parameters to optimize ventilation of all regions while mitigating ventilator-induced lung injury (i.e. barotrauma, volutrauma, atelectrauma, biotrauma).

The primary differentiator of the disclosed approach from utilization of raw scan data only includes, but is not limited to, the ability to determine optimal lung-protective ventilation and personalized treatment guidelines through the addition of flow and fluid-structure interaction simulations. In this regard, the subject technology enables replacement of ‘rule of thumb’ or protocol-driven population settings for tidal volume, maximum pressure, and PEEP with targeted values for the individual patient. Furthermore, results from the disclosed system may be combined with academic knowledge and real-world experience in a practical tool that can greatly benefit patients.

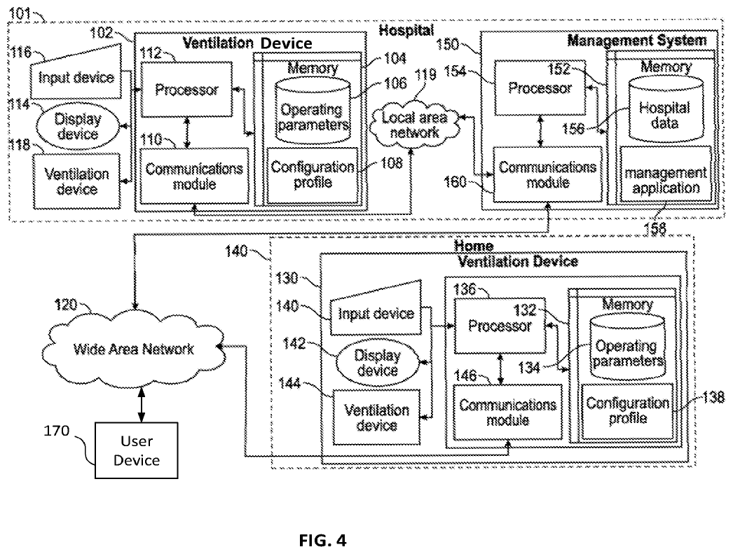

FIG. 4 is a block diagram illustrating an example system for generating patient-specific ventilation settings based on lung modeling, and for adjusting an operation of a ventilator, including a ventilation device, one or more management devices, according to certain aspects of the subject technology. The system may assess conditions of ventilated patients and adjusting an operation mode of a ventilator, including a ventilation device 102, a management system 150, and a ventilation device 130, according to certain aspects of the subject technology. Management system 150 may include a server and, in many aspects, includes logic and instructions for providing the functionality previously described with regard to FIGS. 1 through 3. For example, a server of management system 150 may broker communications between the various devices, and/or generate user interface 10 for display by user devices 170. Ventilation device 102 and ventilation device 130 may represent each of multiple ventilation devices connected to management system 150. Although the management system 150 is illustrated as connected to a ventilation device 102 and a ventilation device 130, the management system 150 is configured to also connect to different medical devices, including infusion pumps, point of care vital signs monitors, and pulmonary diagnostics devices. In this regard, device 102 or device 130 may be representative of a different medical device.

Ventilation device 102 is connected to the management system 150 over the LAN 119 via respective communications modules 110 and 160 of the ventilation system 102 and the management system 150. The management system 150 is connected over WAN 120 to the ventilation device 130 via respective communications modules 160 and 146 of the management system 150 and the ventilation device 130. The ventilation device 130 is configured to operate substantially similar to the ventilation device 102 of a hospital system 101, except that the ventilation device (or medical device) 130 is configured for use in the home 140. The communications modules 110, 160, and 146 are configured to interface with the networks to send and receive information, such as data, requests, responses, and commands to other devices on the networks. The communications modules 110, 160, and 146 can be, for example, modems, Ethernet cards, or WiFi component modules and devices.

The management system 150 includes a processor 154, the communications module 160, and a memory 152 that includes hospital data 156 and a management application 158. Although one ventilation device 102 is shown in FIG. 16, the management system 150 is configured to connect with and manage many ventilation devices 102, both ventilation devices 102 for hospitals and corresponding systems 101 and ventilation devices 130 for use in the home 140.

In certain aspects, the management system 150 is configured to manage many ventilation devices 102 in the hospital system 101 according to certain rules and procedures. For example, when powering on, a ventilation system 102 may send a handshake message to the management system 150 to establish a connection with the management system 150. Similarly, when powering down, the ventilation system 102 may send a power down message to the management system 150 so that the management system 150 ceases communication attempts with the ventilation system 102.

The management system 150 is configured to support a plurality of simultaneous connections to different ventilation devices 102 and ventilation devices 130, and to manage message distribution among the different devices, including to and from a user device 170. User device 170 may be a mobile device such as a laptop computer, tablet computer, or mobile phone. User device 170 may also be a desktop or terminal device authorized for use by a user. In this regard, user device 170 is configured with the previously described messaging application depicted by FIGS. 1 through 15 to receive messages, notifications, and other information from management system 150, as described throughout this disclosure.

The number of simultaneous connections can be configured by an administrator in order to accommodate network communication limitations (e.g., limited bandwidth availability). After the ventilation device 102 successfully handshakes with (e.g., connects to) the management system 150, the management system 150 may initiate communications to the ventilation device 102 when information becomes available, or at established intervals. The established intervals can be configured by a user so as to ensure that the ventilation device 102 does not exceed an established interval for communicating with the management system 150.

The management system 150 can receive or provide data to the ventilation device 102, for example, to adjust patient care parameters of the ventilation device. For instance, alerts may be received from ventilation device 102 (or device 130) responsive to thresholds being exceeded. An admit-discharge-transfer communication can be sent to specified ventilation devices 102 within a certain care area of a hospital 101. Orders specific to a patient may be sent to a ventilation device 102 associated with the patient, and data specific to a patient may be received from ventilation device 102.

The ventilation device 102 may initiate a communication to the management system 150 if an alarm occurs on the ventilation system 102. The alarm may be indicated as time-sensitive and sent to the beginning of the queue for communicating data to the management system 150. All other data of the ventilation device 102 may be sent together at once, or a subset of the data can be sent at certain intervals.

Hospital data 156 may continuously or periodically received (in real time or near real time) by management system 150 from each ventilator device 102 and each ventilator device 130. The hospital data 156 may include configuration profiles configured to designate operating parameters for a respective ventilation device 102, operating parameters of each ventilation device 102 and/or physiological statistics of a patient associated with the ventilation device 102. Hospital data 156 also includes patient data for patients admitted to a hospital or within a corresponding hospital system 101, order (e.g., medication orders, respiratory therapy orders) data for patients registered with the hospital 101 system, and/or user data (e.g., for caregivers associated with the hospital system 101). As described previously with regard to the systems described with regard to FIGS. 1 through 7, hospital data 156 may be updated or changed based on an updated state provided by these systems.

The physiological statistics and/or measurements of the ventilator data includes, for example, a statistic(s) or measurement(s) indicating compliance of the lung (Cdyn, Cstat), flow resistance of the patient airways (Raw), inverse ratio ventilation (I/E), spontaneous ventilation rate, exhaled tidal volume (Vte), total lung ventilation per minute (Ve), peak expiratory flow rate (PEFR), peak inspiratory flow rate (PIFR), mean airway pressure, peak airway pressure, an average end-tidal expired CO2 and total ventilation rate. The operating parameters include, for example, a ventilation mode, a set mandatory tidal volume, positive end respiratory pressure (PEEP), an apnea interval, a bias flow, a breathing circuit compressible volume, a patient airway type (for example endotracheal tube, tracheostomy tube, face mask) and size, a fraction of inspired oxygen (FiO2), a breath cycle threshold, and a breath trigger threshold.

The processor 154 of the management system 150 is configured to execute instructions, such as instructions physically coded into the processor 154, instructions received from software (e.g., management application 158) in memory 152, or a combination of both. For example, the processor 154 of the management system 150 executes instructions to receive ventilator data from the ventilation device(s) 102 (e.g., including an initial configuration profile for the ventilation system 102).

Ventilation device 102 is configured to send ventilator information, notifications (or “alarms”), scalars, operating parameters 106 (or “settings”), physiological statistics (or “monitors”) of a patient associated with the ventilation device 102, and general information. The notifications include operational conditions of the ventilation device 102 that may require operator review and corrective action. Scalars include parameters that are typically updated periodically (e.g., every 500 ms) and can be represented graphically on a two-dimensional scale. The physiological statistics represent information that the ventilation device 102 is monitoring, and can dynamic based on a specific parameter. The operating parameters 106 represent the operational control values that the caregiver has accepted for the ventilation device 102. The general information can be information that is unique to the ventilation device 102, or that may relate to the patient (e.g., a patient identifier). The general information can include an identifier of the version and model of the ventilation device 102. It is also understood that the same or similar data may be communicated between management system 150 and ventilation device 130.

FIG. 4 is a block diagram illustrating an example system for generating patient-specific ventilation settings based on lung modeling, and for adjusting an operation of a ventilator, including a ventilation device, one or more management devices, according to certain aspects of the subject technology. Management system 150 may include (among other equipment) a centralized server and at least one data source (e.g., a database 152). The centralized server and data source(s) may include multiple computing devices distributed over a local 119 or wide area network 120, or may be combined in a single device. Data may be stored in data source(s) 152 (e.g., a database) in real time and managed by the centralized server. In this regard, multiple medical devices 102, 130 may communicate patient data, over network 119, 120, to the centralized server in real time as the data is collected or measured from the patient, and the centralized server may store the patient data in data source(s) 152. According to some implementations, one or more servers may receive and store the patient data in multiple data sources.

According to various implementations, management system 150 (including centralized server) is configured to (by way of instructions) generate and provide virtual user interface 10 to clinician devices 170. In some implementations, management system 150 may function as a web server, and virtual interface 100 may rendered from a website provided by management system 150. According to various implementations, management system 150 may aggregate real time patient data and provide the data for display in virtual interface 100. The data and/or virtual interface 100 may be provided (e.g., transmitted) to each clinician device 170, and each clinician device 170 may include a software client program or other instructions configured to, when executed by one or more processors of the device, render and display virtual interface 100 with the corresponding data. The depicted clinician devices 170 may include personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity. While not shown in FIG. 16, it is understood that the connections between the various devices over local network 119 or wide area network 120 may be made via a wireless connection such as WiFi, BLUETOOTH, Radio Frequency, cellular, or other similar connection.

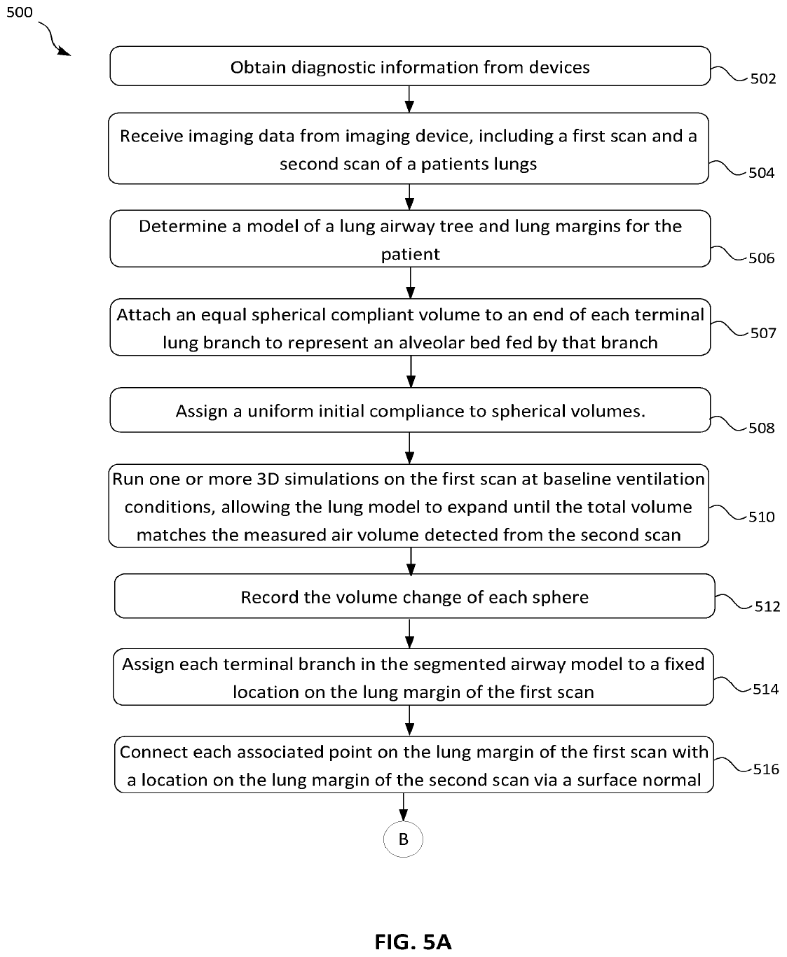

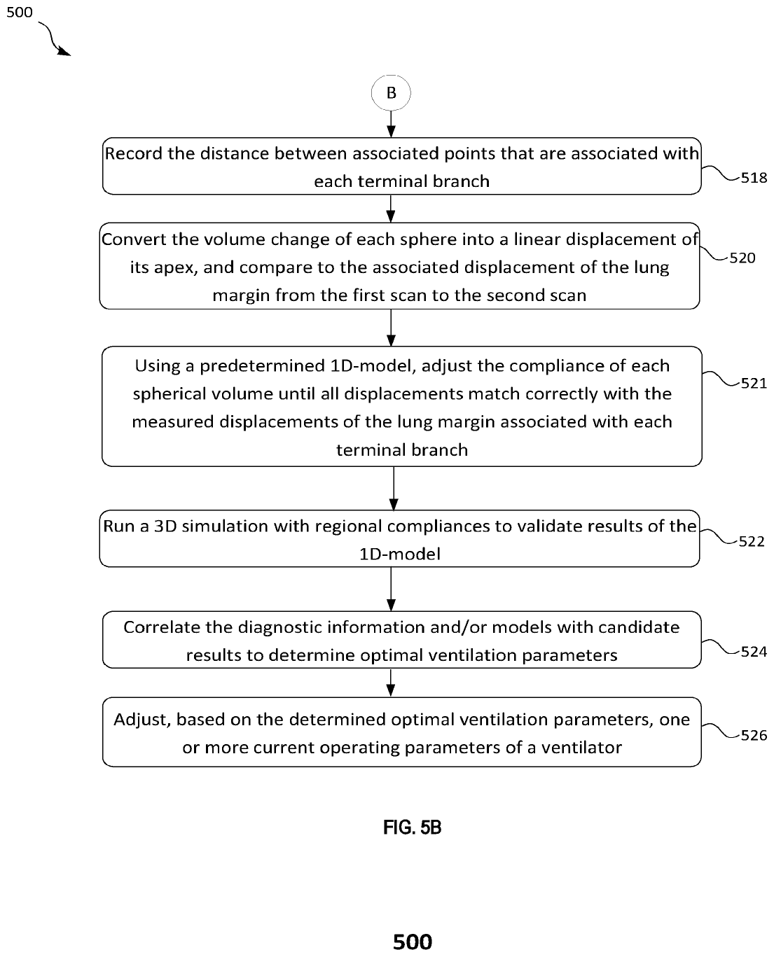

FIGS. 5A and 5B depict an example flow chart of a method of generating patient-specific ventilation settings based on lung modeling, and for adjusting an operation of a ventilator, according to aspects of the subject technology. The process 500 is implemented, in part, through the exchange of data between the ventilation device 102, the management system 150, and user device 170. For explanatory purposes, the various blocks of example process 500 are described herein with reference to FIGS. 1 through 4, and the components and/or processes described herein. The one or more of the blocks of process 500 may be implemented, for example, by a computing device, including a processor and other components utilized by the device. In some implementations, one or more of the blocks may be implemented apart from other blocks, and by one or more different processors or devices. Further for explanatory purposes, the blocks of example process 500 are described as occurring in serial, or linearly. However, multiple blocks of example process 500 may occur in parallel. In addition, the blocks of example process 500 need not be performed in the order shown and/or one or more of the blocks of example process 500 need not be performed.

The example process may be implemented by a system comprising a ventilation communication device configured to receive ventilation data, a medication delivery communication device configured to receive medication delivery information associated with an ongoing administration of a medication to the patient, an image capture device (e.g., an X-Ray, CT or MRI scanning system), and one or more sensors configured to obtain physiological data from a patient. The disclosed system may include a memory storing instructions and data, and one or more processors configured to execute the instructions to perform operations.

In the depicted example flow diagram, certain information is (optionally) obtained from the various component devices (502). According to some implementations, diagnostic information is received for the patient by the management system 150, and the management system 150 determines, based on signals received from the one or more sensors, a physiological state of the patient. In some implementations, system 150 determines, from the ventilation communication device, an operational mode of the ventilator. System 150 may (optionally) receive medication delivery information from the medication delivery communication device.

System 150 receives imaging data from an imaging device (CT scanner) (504). In the depicted process, two CT scans of a patient's lungs are received into a storage memory. A first scan may be taken at a breath-hold at the end of exhalation and a second scan may be taken at a breath-hold at the end of inhalation. In some implementations, the image data may include MRI data.

System 150 determines, based on the image data, a model of a lung airway tree and lung margins (envelope perimeter) of the patient (506). According to various implementations, system 150 may generate a respective model for each scan. As depicted in FIG. 3, the system attaches a (e.g. an equal) spherical compliant volume to the end of each terminal lung branch (bronchiole) to represent one or more alveoli (e.g. an alveolar bed) fed by that branch (507). According to various implementations, the attached spherical compliant volume is a virtual model of one or more alveoli at the end of one or more lung branches (e.g. each being an air chamber that can stretch) with a default compliance value (e.g. a value corresponding to its elasticity). A spherical compliant volume has the ability to expand (and/or change shape) as an amount of gas contained by the volume increases according to its given compliance value. According to various implementations, the initial volume of each spherical compliant volume (e.g. sphere) is determined by the measured air volume from the first scan minus the volume of the segmented airway tree, divided by the total number of terminal branches. According to some implementations, the model and/or the spherical compliant volume may also be determined and or modified, at least in part, based on the received diagnostic information.

System 150 assigns a uniform initial compliance to all spherical volumes (508), and runs 3D FSI simulations on the (model of the) first scan at baseline ventilation conditions, allowing the lung model to expand until the total volume matches the measured air volume detected from (a model of) the second scan (510). In this regard, the model of the second scan may be used to determine a volume (as described previously) at the breath-hold at the end of inhalation (e.g. representative of a maximum air volume). The volume change of each spherical compliant volume (e.g., ratio Vol2/Vol1 per sphere) is recorded (512), and system 150 assigns each terminal branch in the segmented airway model to a fixed location on the lung margin of the first scan (514). System 150 may automatically determine the location by extending branch to nearest location on perimeter.

As depicted in FIG. 3 (as a green line), system 150 connects each associated point on the lung margin of the first scan with a location on the lung margin of the second scan via surface normal (516). System 150 records the distance between associated points (e.g., the length of the green line) associated with each terminal branch (e.g., terminal bronchiole) (518). System 150 converts the volume change of each spherical compliant volume into a first linear displacement of its apex from a starting point to its point in an expanded state, and compares, for each spherical compliant volume, the first linear displacement to an associated displacement of the lung margin from the first scan to the second scan at a location associated with the displaced apex (520).

In other words, a point is selected on a surface of the spherical compliant volume when in an initial state (e.g. a point on a surface of the sphere at a first volume), and the linear distance between that point and the same point when the volume is in an expanded state is measured to determine the linear displacement. A second point on a surface of the lung (e.g. the lung margin) corresponding to each spherical compliant volume is determined and a second linear displacement (or distance) between that second point in an initial state and that same second point in the lung's expanded state is determined. The initial state of the lung margin may correspond to the first image scan (or model of the scan) and the lung's expanded state may correspond to the second image scan (or model of the scan). System 150 (and/or a corresponding algorithm) may be preprogramed to index point locations between respective alveoli (and spherical compliant volumes) and respective point locations on a scan or model of the patient's lung, or may utilize artificial intelligence and image recognition to determine the index automatically. Determination of lung margin and the forgoing analysis may be made based on two-dimensional image scans or three-dimensional models. Optionally, an optimization may be performed. In this regard, system 150, using a predetermined model (e.g. a one, two or three dimensional model), adjusts the compliance of each spherical volume until all displacements match correctly with the measured displacements of the lung margin associated with each terminal branch (521). In this regard, system 150 determines how much each respective spherical compliant volume (or sphere) moved with how much a corresponding surface of the lung moved. The compliance value of the volume is adjusted so that the first linear displacement matches the corresponding second linear displacement of the lung. The adjustment may be incremental over multiple spherical compliant volumes for a given location of a lung, and processed iteratively to obtain a final result. That is, the adjustment(s) may be performed iteratively, each time reperforming the foregoing comparison, until the comparing of each first linear displacement of a spherical compliant volume is consistent with the corresponding second linear displacement of the lung margin. System 150 may not render a regional compliance map for the lungs. System 150 runs a 3D FSI simulation with regional compliances to validate results of the 1D-model (522). System 150 runs one or more simulations with 1D-model to establish appropriate ventilation parameters to optimize ventilation of all regions while mitigating ventilator-induced lung injury (e.g., barotrauma, volutrauma, atelectrauma, biotrauma).

According to some implementations, the foregoing modeling, calculations and/or determinations may be facilitated, at least in part, by a neural network. For example, system 150 may provide the determined physiological state of the patient, the determined physical state of the patient, the determined operational mode of the ventilator, the medication delivery information, and the received diagnostic information for the patient to a neural network, and receives, from the neural network, the lung model. The neural network may further be used to correlate the received data and/or the generated models with candidate results to determine optimal ventilation parameters (524). For example, system 150 may receive candidate results, for example from a healthcare information system. The results may be for several or a multitude (e.g. more than 100) patients. Each candidate result may include patient diagnostic parameters for a patient, for example, diagnostic information including lung volume or physiological state of the patient, imaging data, or other data that may be used to construct one or more lung models as previously described for the patient. Each candidate result may further include a lung treatment outcome for the patient after a period of ventilation treatment, and corresponding ventilation parameters that were used to treat the patient during the ventilation treatment. Optimal ventilation parameters are those parameters utilized during a ventilation that resulted in a positive lung treatment outcome. For example, the patient was weaned off of ventilation earlier than a population of other patients (e.g. a majority) and/or more than a threshold number (e.g. a majority) of measured physiological parameters of the patient recovered to normal values earlier than the population. After the results are correlated, the system 150 adjusts, based on the determined optimal ventilation parameters, one or more current operating parameters of the ventilator 102, 130 to influence the operational mode of the ventilator (526). In this manner, patient-specific ventilation modes and settings may be generated by the system to optimize oxygenation, mitigate lung injury and expedite weaning of the patient from ventilation.

According to various implementations, physiogical data may be received from one or more sensors. The sensors may include a sensor configured to obtain a vital sign measurement of the patient, including one or more of blood pressure, patient core temperature, heart rate, electrocardiogram (ECG) signal, pulse, or blood oxygen saturation level, wherein the determined physiological state of the patient comprises information representative of the vital sign measurement. The sensors may include a sensor applied to the patient's skin and configured to measure a level of muscle tension. In some implementations, the medication delivery communication device (e.g., component 14) is configured to receive, from an infusion pump, the medication delivery information, the medication delivery information comprising a drug identification, drug concentration, drug dosage, or length of an ongoing infusion. In some implementations, management system 150 (or hospital system 101) is configured to receive diagnostic information for the patient. The diagnostic information may include lab results associated with the patient received from a diagnostic information system.

Many aspects of the above-described example 900, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

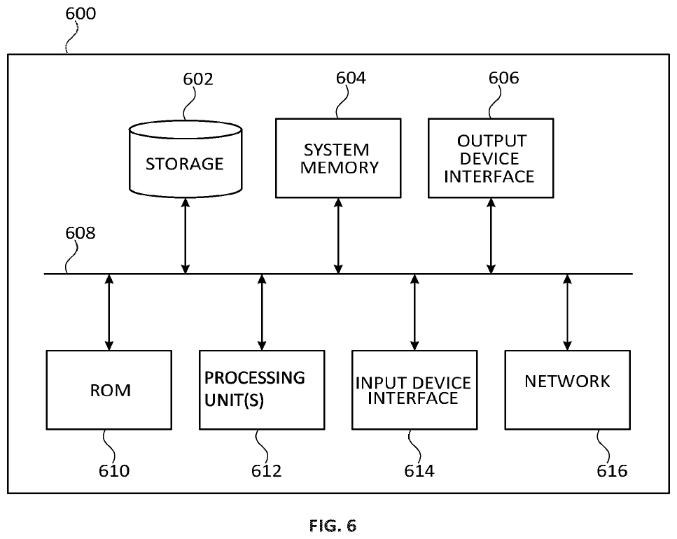

FIG. 6 is a conceptual diagram illustrating an example electronic system for generating patient-specific ventilation settings based on lung modeling, and for adjusting an operation of a ventilator, according to aspects of the subject technology. Electronic system 600 may be a computing device for execution of software associated with one or more portions or steps of process 600, or components and processes provided by FIGS. 1 through 5. Electronic system 600 may be representative, in combination with the disclosure regarding FIGS. 1 through 9, of the management system 150 (or server of system 150) or the clinician device(s) 170 described above. In this regard, electronic system 600 or computing device may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 600 may include various types of computer readable media and interfaces for various other types of computer readable media. In the depicted example, electronic system 1700 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and processes previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves instructions to execute and data to process in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such a random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 10, bus 608 also couples electronic system 1700 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 1700 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself

As used in this specification and any claims of this application, the terms “computer,” “server,” “processor,” and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML, page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method for generating patient-specific ventilation settings to adjust an operation mode of a ventilator providing ventilation to a patient, comprising: receiving diagnostic information for a patient, the diagnostic information including a first image scan and a second image scan of the patient's lungs; determining, based on the diagnostic information, a first lung model of a lung airway tree and lung margins for the patient, the lung airway tree comprising a plurality of lung branches; generating and assigning a spherical compliant volume to an end of each lung branch of the lung airway tree to represent an alveolar bed fed by the lung branch; executing a three-dimensional simulation on the determined first lung model at a baseline ventilation condition, allowing each spherical compliant volume of the first lung model to expand from an initial state to an expanded state in which a total volume of the first lung model matches a measured air volume determined from a second lung model of the second image scan; determining a volume change of each spherical compliant volume based on the expanding of the spherical compliant volume from the initial state to the expanded state; assigning each spherical compliant volume in the first lung model to a fixed location on the lung margin of the first lung model associated with the first image scan; connecting respective points on the lung margin of the first lung model with respective points on the lung margin of the second lung model of the second image scan; converting the determined volume change of each spherical compliant volume into a first linear displacement between a starting point on the spherical compliant volume while in the initial state to the same point in the expanded state; comparing, for each spherical compliant volume, the first linear displacement of the spherical compliant volume to a second linear displacement of the corresponding fixed location on the lung margin measured between the first image scan and the second image scan; adjusting a compliance value of each spherical volume based on the comparing until the first linear displacement of each sphere corresponds to the second linear displacement measured for the corresponding fixed location of the lung margin; updating the first lung model of the lung airway tree based on the adjusting of the compliance value of each spherical volume; receiving a plurality of candidate results, each result including patient diagnostic parameters for a patient, a lung treatment outcome for the patient after a period of ventilation treatment, and corresponding ventilation parameters for the ventilation treatment; correlating the updated first lung model with the received candidate results to determine optimal ventilation parameters; and adjusting, based on the determined optimal ventilation parameters, one or more current operating parameters of the ventilator to provide ventilation to the patient.

Clause 2. The method of Clause 1, wherein the predetermined model is a one-dimensional model, the method further comprising: running a three-dimensional simulation with regional compliances to validate results of the one-dimensional model.

Clause 3. The method of Clause 1, wherein the optimal ventilation parameters are further determined based on correlating the received diagnostic information with the candidate results.

Clause 4. The method of Clause 1, wherein the respective points on the lung margin of the first lung model are connected with respective points on the lung margin of the second lung model of the second image scan using a surface normal value.

Clause 5. A system, comprising: a ventilation communication device configured to receive ventilation data; a medication delivery communication device configured to receive medication delivery information associated with an ongoing administration of a medication to a patient; an image capture device; one or more sensors; a memory storing instructions; and one or more processors configured to execute the instructions to perform the method of Clause 1.

### Further Consideration

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit this disclosure.

The term website, as used herein, may include any aspect of a website, including one or more web pages, one or more servers used to host or store web related content, etc. Accordingly, the term website may be used interchangeably with the terms web page and server. The predicate words “configured to,” “operable to,” and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

1. A method for generating ventilation settings to adjust an operation mode of a ventilator, comprising:
receiving diagnostic information for a patient, the diagnostic information including a first image scan and a second image scan of the patient's lungs;
determining, based on the diagnostic information, a first lung model of a lung airway tree and lung margins for the patient, the lung airway tree comprising a plurality of lung branches;
generating and assigning a spherical compliant volume to an end of each lung branch of the lung airway tree to represent one or more alveoli fed by the lung branch;
executing a three-dimensional simulation on the determined first lung model at a baseline ventilation condition, allowing each spherical compliant volume of the first lung model to expand from an initial state to an expanded state in which a total volume of the first lung model matches a measured air volume determined from a second lung model of the second image scan;
determining a volume change of each spherical compliant volume based on the expanding of the spherical compliant volume from the initial state to the expanded state;
assigning each spherical compliant volume in the first lung model to a fixed location on the lung margin of the first lung model associated with the first image scan;
connecting respective points on the lung margin of the first lung model with respective points on the lung margin of the second lung model of the second image scan;
converting the determined volume change of each spherical compliant volume into a first linear displacement between a starting point on the spherical compliant volume while in the initial state to the same point in the expanded state;
comparing, for each spherical compliant volume, the first linear displacement of the spherical compliant volume to a second linear displacement of the corresponding fixed location on the lung margin measured between the first image scan and the second image scan;
adjusting a compliance value of each spherical volume based on the comparing until the first linear displacement of each sphere corresponds to the second linear displacement measured for the corresponding fixed location of the lung margin;
updating the first lung model of the lung airway tree based on the adjusting of the compliance value of each spherical volume;
receiving a plurality of candidate results, each result including patient diagnostic parameters for a patient, a lung treatment outcome for the patient after a period of ventilation treatment, and corresponding ventilation parameters for the ventilation treatment;
correlating the updated first lung model with the received candidate results to determine optimal ventilation parameters; and
adjusting, based on the determined optimal ventilation parameters, one or more current operating parameters of the ventilator.

2. The method of claim 1, wherein the predetermined model is a one-dimensional model, the method further comprising:
running a three-dimensional simulation with regional compliances to validate results of the one-dimensional model.

3. The method of claim 1, wherein the optimal ventilation parameters are further determined based on correlating the received diagnostic information with the candidate results.

4. The method of claim 1, wherein the respective points on the lung margin of the first lung model are connected with respective points on the lung margin of the second lung model of the second image scan using a surface normal value.

5. A system, comprising:
a ventilation communication device configured to receive ventilation data;
a medication delivery communication device configured to receive medication delivery information associated with an ongoing administration of a medication to a patient;
an image capture device;
one or more sensors;
a memory storing instructions; and
one or more processors configured to execute the instructions to perform the method of claim 1.
